(19) United States
(12) Patent Application Publication
(10) Pub. No.: US 2024/0274235 A1
(43) Pub. Date: Aug. 15, 2024

(54) METHOD OF MULTIPLE SEQUENCE ALIGNMENT USING GRADIENT DESCENT

(71) Applicant: Daniel Kim, Chappaqua, NY (US)

(72) Inventor: Daniel Kim, Chappaqua, NY (US)

(21) Appl. No.: 18/645,380

(22) Filed: Apr. 25, 2024

(51) Int. Cl. G16B 30/10

(52) U.S. Cl. CPC G16B 30/10

(57) ABSTRACT

Embodiments of the invention are directed to a method for aligning multiple sequences that models a sequence as a series of bases, wherein adjacent bases are connected through flexible spaces with variable lengths that represent extra spaces between adjacent bases. The method begins with creating an array of floating-point variables that represent spaces for bases within sequences. The method iteratively converges to the optimum set of spaces by gradient descent. In a non-limiting example of the method, the gradients of a space can be obtained by calculating a cost value used to increment or decrement the corresponding space by an integer amount. After each iteration, the spaces are updated by summing the calculated gradients scaled by a change rate, and the sequences are updated by adding or removing gaps between each base according to the quantized spaces. The final sequences become the solution to the multiple sequence alignment task.

### BACKGROUND

The present invention relates to a method of aligning multiple sequences, wherein each sequence is composed of a set of predefined symbols. Examples of such sequences exist in biology through DNA and RNA, which store sequential genetic information wherein the symbols are nitrogenous bases, and in natural language, wherein the symbols are letters or words.

In fields such as bioinformatics, sequences are often compared to identify regions of similarity that may indicate functional, structural, or evolutionary relationships between the sequences. For proper comparison, sequences should first be properly aligned. The alignment involves more than just shifting one sequence versus others; it also requires inserting gaps into the sequences to compensate for indels (insertion or deletion of one or more bases).

Several different methods to find an optimal or near-optimal alignment of two or more sequences have been developed. They include dynamic programming, progressive, and iterative methods. Conventional methods have a trade-off between an alignment's accuracy and the cost of its computation—i.e., computing time and computing resources such as memory capacity. The computing cost increases dramatically as either the sequence length or the number of sequences increases.

In conventional alignment methods, alignments are adjusted for individual sequences one at a time or on a pair-wise basis, which makes multiple sequence alignment (MSA) inefficient and challenging. Different interim alignments may be tried and searched in a combinatorial manner, but it is almost impossible to search across all the possible combinations. When the number of sequences increases, alignments have relied more and more on approximation so that the procedure can complete in a reasonable time, undermining alignment accuracy.

In this invention, a multiple sequence alignment (MSA) method is described in which alignments of all the input sequences are considered concurrently and adjusted in parallel so that the quality of alignment is maximized while still minimizing the total alignment time by avoiding an exhaustive, time-consuming search across combinatorial space of possible alignments.

### SUMMARY

Embodiments of the invention are directed to a method for aligning multiple sequences that models a sequence as a series of bases, wherein adjacent bases are connected through flexible spaces with variable lengths that represent extra spaces between adjacent bases. The modeling can be also interpreted as a train model with multiple cars that are connected through flexible gangways. The method begins with creating an array of floating-point variables that represent spaces for each base within each sequence. The method iteratively converges to the optimum set of the spaces by gradient descent.

In a non-limiting example of the method, the gradients of a space can be obtained by calculating a cost value used to increment or decrement the corresponding space by an integer amount (for example, +1 or −1). In a non-limiting example of the method, the cost can be a summation of pair-wise alignment scores multiplied by minus 1.

After each iteration, the spaces are updated by adding the calculated gradients multiplied by a given change rate, and the sequences are also updated by adding or removing a number of gaps between each bases according to the updated spaces after quantization. After a predetermined number of iterations, the resulting sequences become the solution to the multiple sequence alignment task.

Additional technical features and benefits are provided by the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1A depicts an example of modeling two input sequences to the train model based on the invention;

FIG. 1B depicts an example of intermediate values for the spaces and correspondingly updated sequences;

FIG. 2A through FIG. 2E depict an exemplary procedure for the first few steps to calculate gradients and update sequences;

FIG. 3 depicts a flow chart that shows one embodiment of the entire procedure of multiple sequence alignment based on the invention;

FIG. 4 depicts a flow chart that shows another embodiment of the entire procedure of multiple sequence alignment based on the invention;

FIG. 5 depicts an example of a conventional guide tree to supplement the explanation for FIG. 6;

FIG. 6 depicts one embodiment of the score calculation procedure that reflects guide tree information;

FIG. 7 depicts a flow chart that shows another embodiment of the entire procedure of multiple sequence alignment assisted by a guide tree based on the invention;

In the accompanying figures and following detailed description of the described embodiments of the invention, the various elements illustrated in the figures are provided with two to four-digit reference numbers. With minor exceptions, the leftmost digit(s) of each reference number correspond to the figure in which its element is first illustrated.

### DETAILED DESCRIPTION

The term “exemplary” is used herein to mean “serving as an example, instance, or illustration.” Any embodiment described herein as “exemplary” is not necessarily to be construed as preferred or advantageous over other embodiments. All of the embodiments described in this Detailed Description are exemplary embodiments provided to enable persons skilled in the art to make or use the invention and not to limit the scope of the invention which is defined by the claims.

It is understood in advance that although exemplary embodiments of the invention are described in connection with DNA (deoxyribonucleic acid), embodiments of the invention are not limited to the types of sequences described in this specification. Rather, embodiments of the present invention are capable of being implemented in conjunction with any other type of genetic or biological sequence including but not limited to RNA (ribonucleic acid) or proteins, or non-biological sequences such as natural language or financial data.

For the sake of brevity, conventional techniques related to sequence alignment may not be described in detail herein. Moreover, the various tasks and process steps described herein can be incorporated into a more comprehensive procedure or process having additional steps or functionality not described in detail herein. Various steps in sequence extraction, alignment scoring, computer modeling and simulation are well known, so, in the interest of brevity, many conventional steps will only be mentioned briefly herein or will be omitted entirely without providing their well-known details.

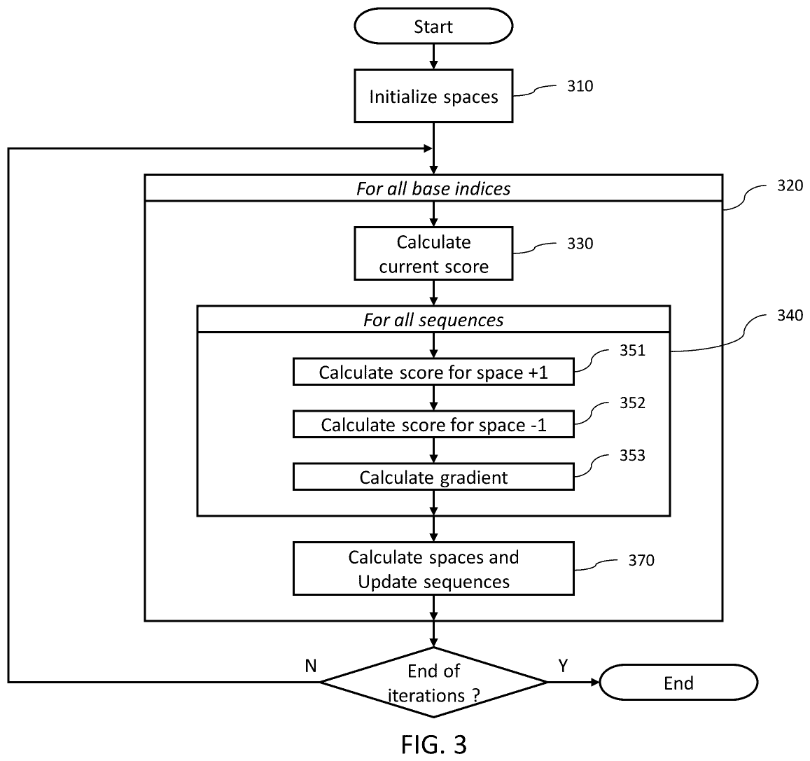

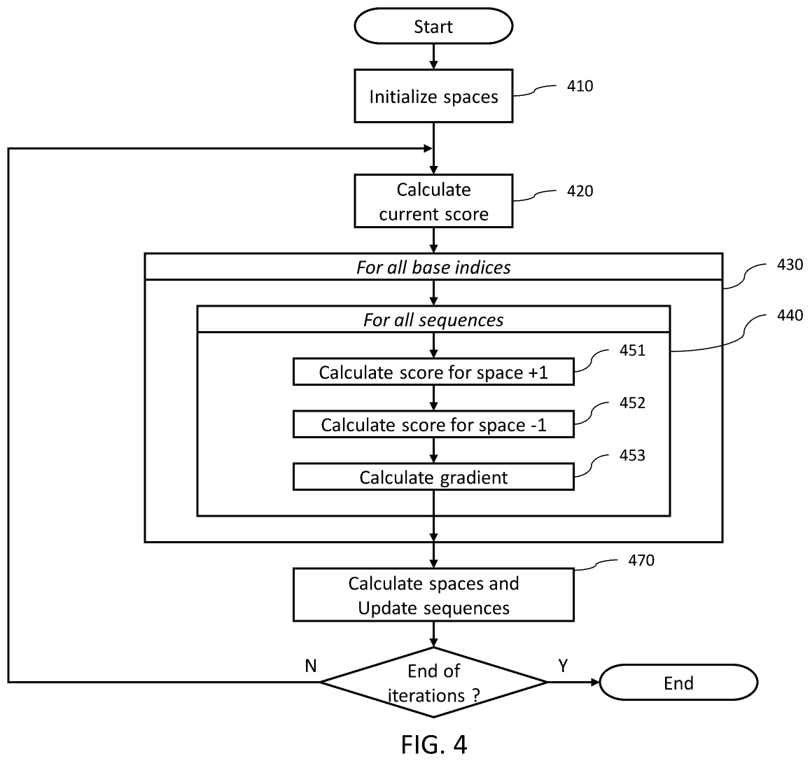

Turning now to an overview of the aspects of the present invention, one or more embodiments of the invention address the aforementioned shortcomings by the following:
1) Spaces for one (per FIG. 3) or all (per FIG. 4) bases in all sequences are updated concurrently in each step. This significantly reduces the total required alignment time, especially for large numbers of sequences.
2) Changes in spaces in each step are smaller than one base length throughout the process. This allows the use of gradient descent to find the optimum solution by making incremental changes that are proportional to the gradient tensor with enough accuracy.

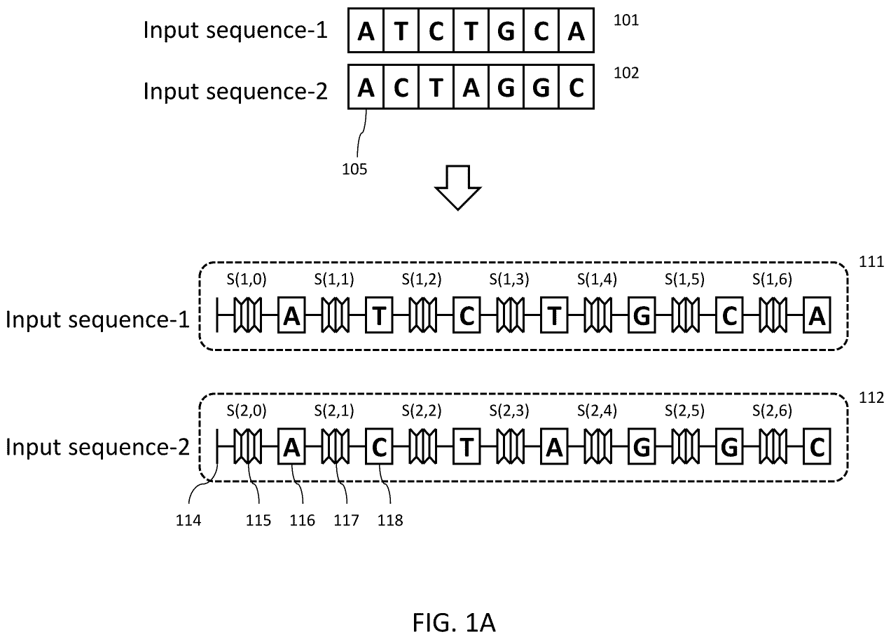

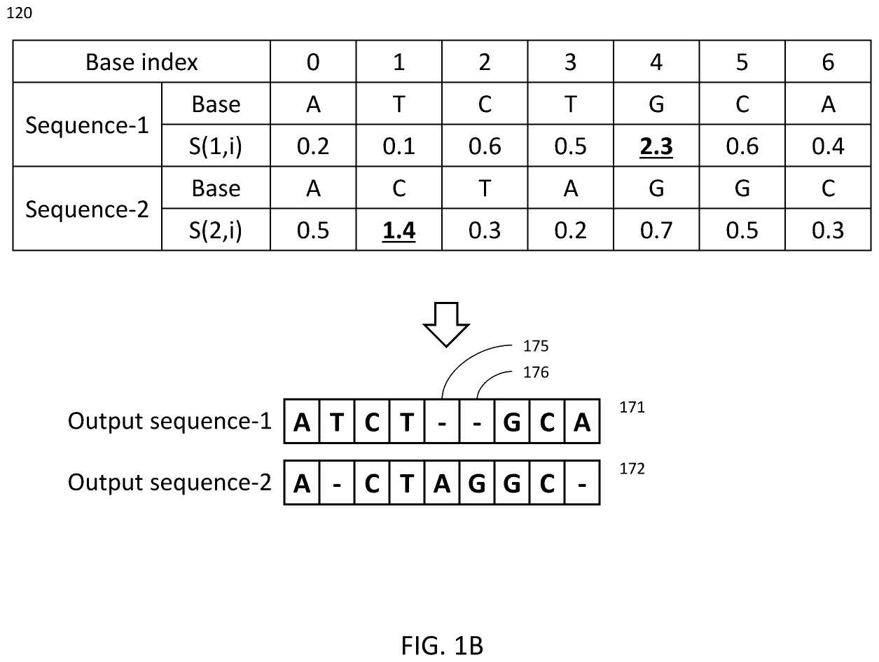

Turning now to a more detailed description of the method according to aspects of the invention, FIG. 1 depicts an example of how two input sequences are modeled according to the train model based on the invention. Two sequences, 101 and 102, are to be aligned. Each sequence is composed of 7 nucleotide bases; for instance, 105 is one of the bases.

Aligning sequences means inserting one or more gaps in between selected adjacent bases so that the alignment score can be maximized. In this invention, the sizes of gaps at each possible location are defined as variables. Furthermore, the number of gaps is considered a quantization of continuous valued (or floating-point) variables, so that those continuously valued variables can be found by numerical analytic procedures such as gradient descent. The continuous valued variable is called the space in this invention and is modeled to be positioned to the left of each base, i.e., in between the previous base and the current base. The accordion shaped elements (for example, 115 and 117) represent the spaces. S(2,0) represents the space to the left of the 0'th base (116) in the 2^{nd} sequence (112). As 116 is the first base in the sequence, its space (115) is positioned in between the initial position (114) and the current base (116).

FIG. 1B depicts an example of intermediate values for the spaces and the correspondingly updated sequences. Table 120 shows an example of a snapshot of intermediate values for the spaces. As shown in 120, the space values are fractional numbers. The space values to the base gap conversion can be simply

$$gap = INT ( space )$$

which means that any space value less than 1 generates no gap. Any space value that is greater than or equal to 1 and less than 2 generate one gap. Any space value that is greater than or equal to 2 and less than 3 generate two gaps, etc. For example, as S(1,4)=2.3, it generates two gaps (175 and 176).

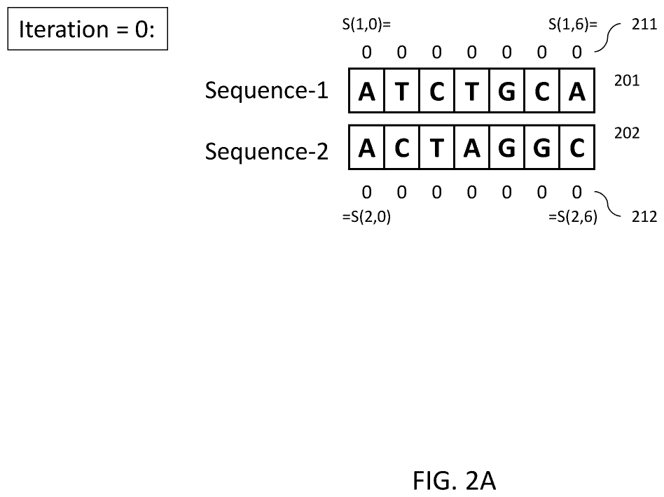

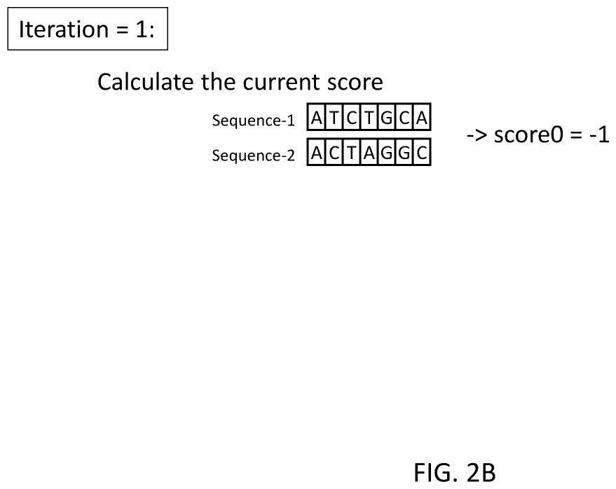

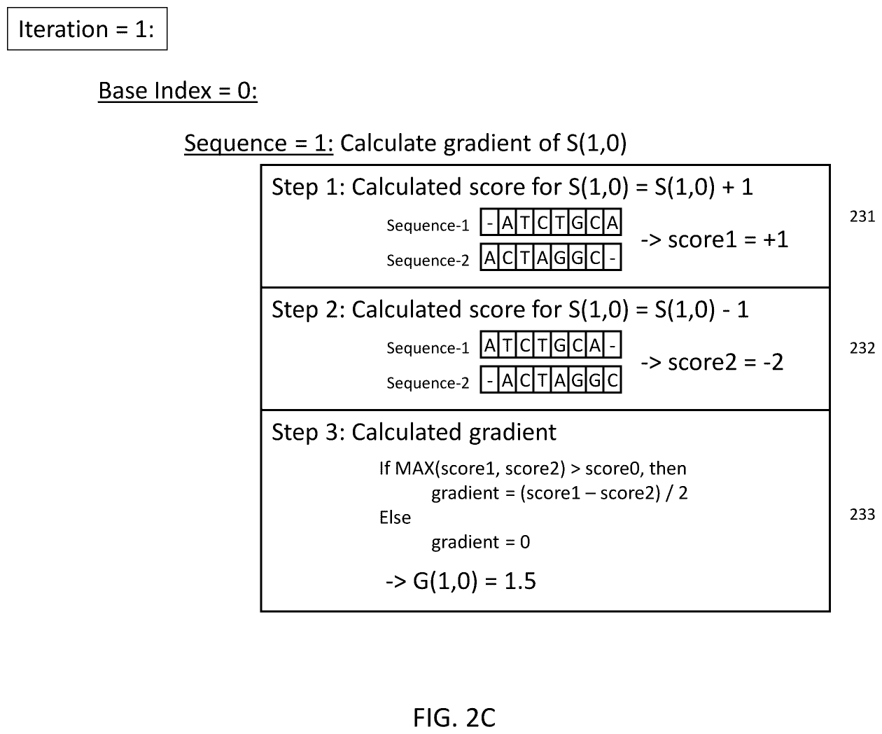

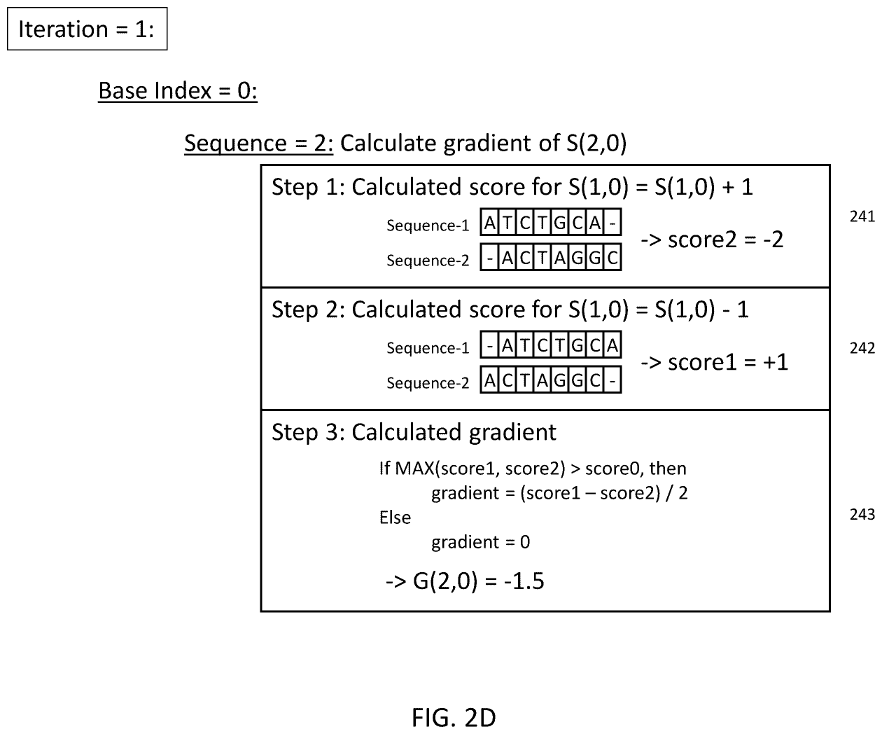

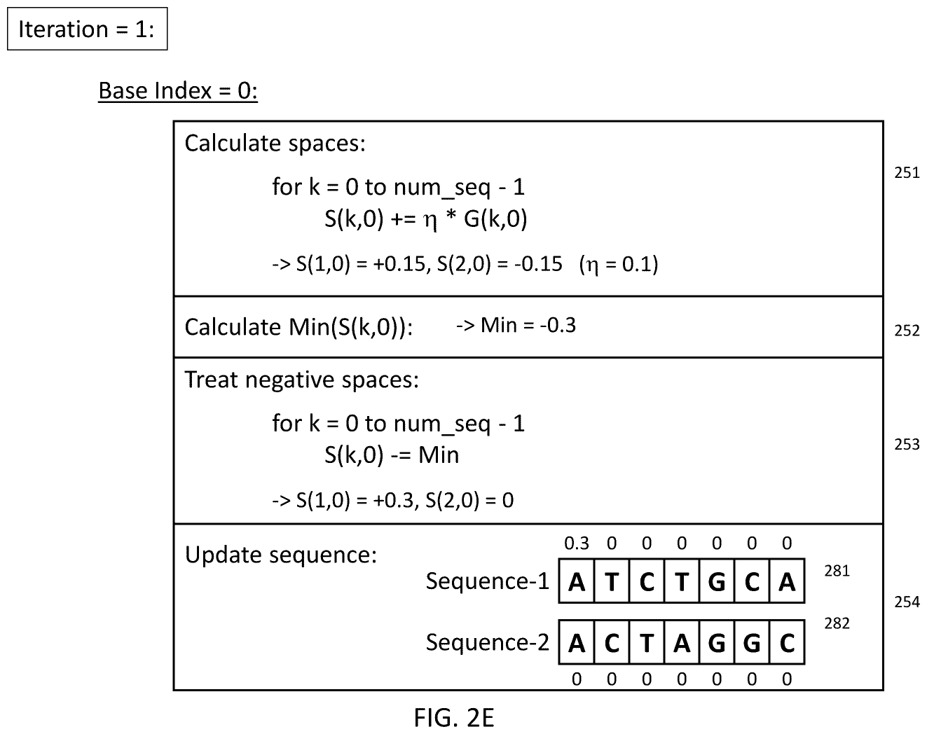

FIG. 2A through 2E depict an exemplary procedure for the first few steps to calculate gradients and update sequences. FIG. 2A shows the initial condition where all the spaces are set to zero. This also corresponds to step 310 in FIG. 3.

FIG. 2B shows the step to calculate the score for the latest sequence states. This score is used as the baseline score to calculate the gradient, or in other words, to decide the direction of changes in the space at the next update. This also corresponds to step 330 in FIG. 3. In this example, the score is calculated to be −1 and is stored as score0. As an example, the following scoring rules were used:

$$\begin{matrix}1 ) Match = \text{+2} \\ 2 ) Mismatch = \text{-1} \\ 3 ) Gap = \text{-1}\end{matrix}$$

In this embodiment, the scores were calculated across all the bases in all the sequences. In another embodiment, the scores can be calculated across the same bases in all the sequences, or across a predetermined number of bases, starting from the current base, in all the sequences.

FIG. 2C depicts the procedure to calculate the gradient for the space of the 0'th base (‘A’) in the 1^{st} sequence, S(1,0). In step 1 (231), the score is calculated for the case that the space is incremented by 1, which is equivalent to inserting a gap to the left of the 0'th base (‘A’) in the 1^{st} sequence. In this example, the score is calculated to be +1, and is stored as score1.

Similarly, in step 2 (232), the score is calculated for the case that the space is decremented by 1, which is equivalent to inserting a gap to the left of the 0'th base (‘A’) in all other sequences, because spaces should not be negative. In this example, the score is calculated to be −2, and is stored as score2.

Finally in step 3 (233), the gradient is determined. If the score is not improved by either incrementing or decrementing the space, the gradient is set to zero. If not, then the gradient is calculated as

$$G ( 1 , 0 ) = \frac{\partial Score}{\partial S ( 1 , 0 )} = \frac{score1 - score 0}{2} = \frac{+ 1 - ( - 2 )}{2} = 1.5$$

FIG. 2D depicts the procedure to calculate the gradient for the space of the 0'th base (‘A’) in the 2^{nd} sequence, S(2,0). However, because there are only two sequences in this example, the gradient is calculated to be the negative of that of the 0'th base in the 1^{st} sequence, i.e., G(2,0)=−G(1,0).

FIG. 2E depicts the procedure to update the spaces and the sequence. As the negative score was chosen as the cost function, the space change has the same sign as the gradient. However, a scaling factor (denoted as n) is multiplied to control the descending rate of the spaces. In one embodiment, the scaling factor can gradually be made smaller after every iteration to more finely converge to the optimum solution.

After updating the spaces, S(1,0) and S(2,0), any negative space should be adjusted to become positive by increasing all other spaces instead. To do this, the minimum (most negative) space value is subtracted from all the space values as shown in 253. The updated sequence is as shown in 254.

FIG. 3 depicts a flow chart that shows one embodiment of the entire procedure of multiple sequence alignment based on the invention as explained in FIG. 2A through FIG. 2E. Although there were only two input sequences in FIG. 2A through FIG. 2E, the same procedure can be applied to more input sequences.

FIG. 4 depicts a flow chart that shows another embodiment of the entire procedure of multiple sequence alignment based on the invention. The difference between FIG. 3 and FIG. 4 is whether the space update is performed for each base sequentially (FIG. 3), or for the entire sequence simultaneously (FIG. 4).

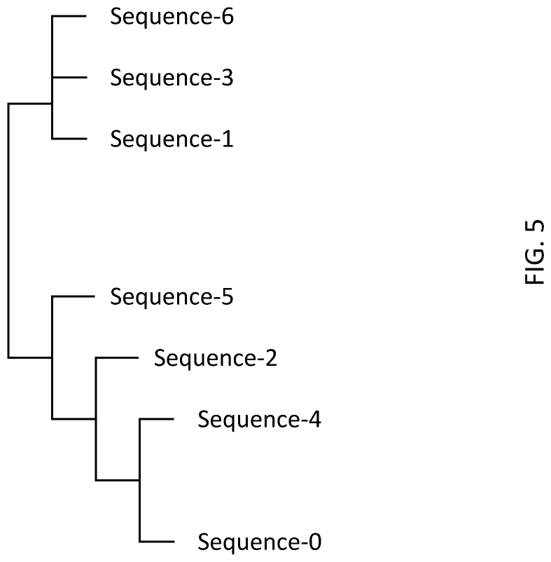

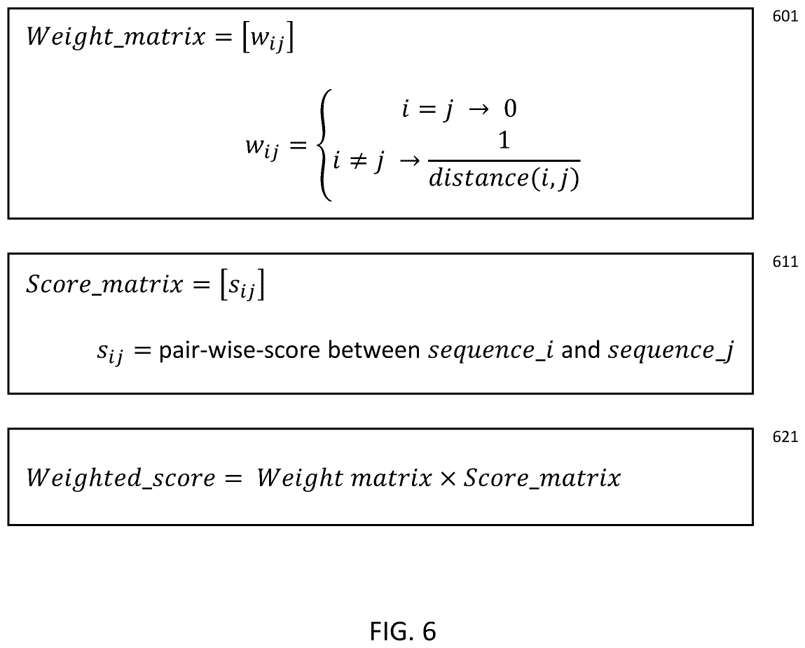

In the embodiments that have been depicted so far, the cost function was chosen to be the negation of the summation of all pair-wise scores. This invention does not preclude the use of any other measure for alignment quality to be used to construct the cost function. One example is to use a weighted score to account for previously determined tree information, such as phylogenetic tree. FIG. 5 depicts an example of a conventional guide tree to supplement explanation for FIG. 6. There are various well-known ways to generate a tree to guide multiple sequence alignment.

FIG. 6 depicts one embodiment of the score calculation procedure that reflects guide tree information. Provided that a guide tree was generated using any known method, a weight matrix can be constructed as in 601. In this embodiment, the weight between two sequences (sequence-i and sequence-j) can be the reciprocal of the distance between two sequences in the tree. The weight score can then be obtained simply by multiplying the weight matrix and the score matrix.

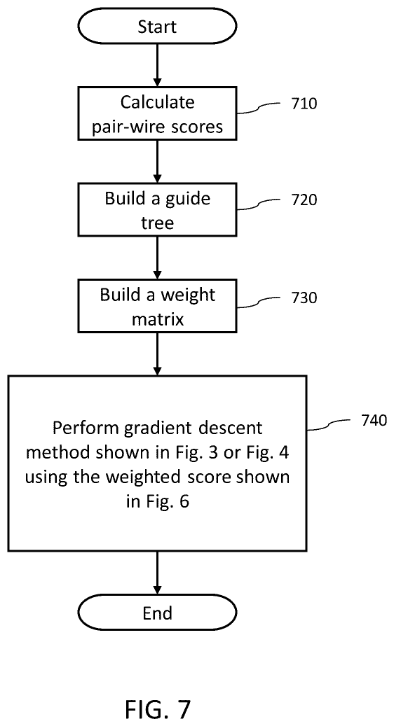

FIG. 7 depicts a flow chart that shows another embodiment of the entire procedure of multiple sequence alignment assisted by a guide tree based on the invention.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

References in the specification to “one embodiment,” “an embodiment,” “an example embodiment,” etc., indicate that the embodiment described can include a particular feature, structure, or characteristic, but every embodiment may or may not include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The flowchart and block diagrams in the Figures illustrate possible implementations of fabrication and/or operation methods according to various embodiments of the present invention. Various functions/operations of the method are represented in the flow diagram by blocks. In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved.

The descriptions of the various embodiments of the present invention have been presented for purposes of illustration but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A method for aligning two or more sequences, the method comprising:
two or more input sequences comprised of a plurality of base elements wherein each element belongs to one of two or more types;
assigning a series of numbers to each element in each of said input sequences wherein said numbers represent the degrees of gaps between adjacent elements in the sequence;
defining a cost function that measures alignment quality;
finding the gradient of the cost function relative to the variation of each of said numbers;
updating the numbers according to the gradient so that the cost function is minimized.

2. The method of claim 1, wherein:
input sequences are biological sequences such as DNA, RNA and proteins.

3. The method of claim 1, wherein:
input sequences are natural language.

4. The method of claim 1, wherein:
input sequences are financial data.

5. The method of claim 1, wherein:
the cost function is an arithmetic function of pair-wise alignment scores.

6. The method of claim 5, wherein:
the cost function is further a function of relative distance in a guide tree.

7. The method of claim 1, wherein:
the number of gaps between elements are quantized value of said numbers.

8. A method for aligning multiple sequences, the method comprising:
multiple input sequences comprised of a plurality of base elements wherein each element belongs to one of two or more types;
mapping input sequences into a system of a train model, the model comprising:
a plurality of trains corresponding to each input sequence;
a plurality of cars in each train corresponding to each base element in a corresponding input sequence;
flexible means of connection between any two adjacent cars wherein the length of each connection represents the gap between base elements;
finding lengths of each connection so that the overall cost function of the train model is minimized;
mapping the train model into output sequences.

9. The method of claim 8, wherein:
lengths are determined iteratively using the process of gradient descent.

10. A method for aligning multiple sequences, comprising:
receiving input sequences;
generating an array of continuous numbers, wherein:
the original numbers are used to calculate the amount they are updated;
the quantized versions of the original numbers are used to determine gaps in updated sequences.
